(19) United States
(12) Patent Application Publication
(10) Pub. No.: US 2012/0185054 A1
(43) Pub. Date: Jul. 19, 2012

(54) MEDIAL PIVOT POSTERIOR STABILIZED KNEE IMPLANT SYSTEM

(75) Inventors: William J. Maloney, Palo Alto, CA (US); John M. Green, Arlington, TN (US); Michael L. Brooks, Brighton, TN (US)

(73) Assignee: WRIGHT MEDICAL TECHNOLOGY, INC., Arlington, TN (US)

(21) Appl. No.: 13/009,117

(22) Filed: Jan. 19, 2011

(51) Int. Cl. A61F 2/38

(52) U.S. Cl. 623/20.28; 623/20.32

(57) ABSTRACT

A knee prosthesis includes a femoral component for mounting to the distal end of the patient's femur and a tibial insert component. The femoral component includes a posterior cam extending between the lateral condylar structure and the medial condylar structure. The tibial insert includes a lateral bearing surface, a medial bearing surface, and a raised post portion having a posterior surface adapted to cooperate with the posterior cam of the femoral component. The posterior surface includes a posteriorlateral camming surface and a posteriormedial camming surface and each of the two camming surfaces have different radii of curvature. Between 60° to 90° of knee flexion, the posterior cam of the femoral component engages the raised post portion of the tibial insert and cooperates with the posteriorlateral and the posterior medial camming surfaces to promote posterior translation of the lateral condylar surface on the lateral bearing surface.

### FIELD OF THE INVENTION

The present disclosure generally relates to knee prostheses that more closely emulate the kinematics of the actual knee joint.

### BACKGROUND

Modern total knee arthroplasty implants replace three separate articulating surfaces within the knee joint: the patello-femoral compartment and the lateral and medial inferior tibio-femoral compartments. Most currently available TKR's are designed to articulate from a position of slight hyperextension to approximately 115° to 130° of knee flexion. A tri-compartmental design can meet the needs of most TKR patients even though the healthy human knee is capable of a range of motion (ROM) approaching 170° of knee flexion. However, there are some TKR patients who have a particular need to obtain high knee flexion in the knee joint. For many, a TKR that permits patients to achieve a ROM in excess of 130° is desirable to allow deep kneeling, squatting and sitting on the floor with the legs tucked underneath.

Conventional total knee replacement implants do not produce normal knee kinematics or motion and generally have a more limited range of motion than a normal knee. This is because conventional total knee replacement implants flex by rotating about a generally horizontal axis during knee flexion and extension, whereas the kinematics of a natural knee joint involve more complex motion of the femur and tibia relative to one another. For example, in a natural knee, the tibia rotates internally about its longitudinal axis during knee flexion; also the medial inferior tibio-femoral compartment exhibits a spinning motion while the lateral inferior tibio-femoral compartment exhibits a rolling motion.

Although some attempts have been made to design a total knee prosthesis which replicates the kinematics of a natural knee, there exists a room for improvement to better replicate the motions previously described.

### SUMMARY

According to an embodiment of the present disclosure, a knee prosthesis for replacement of at least a portion of a knee joint in a leg of a patient is disclosed. The knee prosthesis comprises a femoral component for mounting to the distal end of the patient's femur and a tibial insert component. The femoral component comprises a lateral condylar structure and a medial condylar structure, the lateral condylar structure defining a lateral condylar surface and the medial condylar structure defining a medial condylar surface. The medial condylar surface can be described as a generally spherical surface, extending from the posterior portion of the medial condyle to the anterior portion of the medial condyle. The lateral condylar surface can be described as ovoid, with the posterior portion of the lateral condyle being generally spherical and the anterior portion of the lateral condyle having a surface contour with different radii of curvature in the sagittal (side) and coronal (front) planes. The femoral component also comprises a posterior cam extending between the lateral condylar structure and the medial condylar structure and an anterior cam.

The tibial insert comprises a lateral bearing surface, a medial bearing surface, and a raised post portion having a posterior surface adapted to cooperate with the posterior cam of the femoral component. The medial bearing surface can be described as having a spherical portion with an extended surface in the anterior portion of the bearing surface and an open posterior portion of the medial bearing surface configured to aid in femoral rollback needed to obtain deep knee flexion.

The medial bearing surface is configured to interact with the spherical medial condylar structure of the femoral component to aid in replicating the spinning motion seen in the medial condylar structure in the natural knee. The lateral bearing surface can be described as having a less conforming surface and incorporates the angular excursion necessary for the femoral component to axially rotate about the longitudinal axis as seen in the natural knee. The lateral bearing surface is configured to interact with the ovoid lateral condylar structure of the femoral component to aid in replicating the rolling motion seen in the lateral condylar structure in the natural knee. The raised post portion has posterior surfaces that comprise a posteriorlateral camming surface and a posteriormedial camming surface where each of the two camming surfaces have different radii of curvature when the raised portion interfaces with the posterior cam of the femoral component.

The lateral condylar surface of the femoral component is configured to contact the lateral bearing surface of the tibial insert and the medial condylar surface of the femoral component is configured to contact the medial bearing surface of the tibial insert when the knee prosthesis is installed in the leg of a patient. Prior to engagement of the raised portion of the tibial insert and the posterior cam of the femoral component, the raised spherical anterior lip of the medial compartment of the tibial component interfaces with the spherical surface of the medial condyle of the femoral component to prevent anterior translation of the femoral component and loading of the raised portion of the tibial insert. In a range between 60° to 90° of knee flexion, the posterior cam of the femoral component engages the raised post portion of the tibial insert and promotes a slight posterior translation of the medial condylar surface on the medial bearing surface and greater posterior translation of the lateral condylar surface on the lateral bearing surface.

The knee prosthesis of the present disclosure provides a primary total knee arthroplasty implant that is stable in the primary areas of gait while permitting the patient to achieve deep knee flexion (flexion angles greater than 120°). The knee prosthesis is stable throughout its functional flexion by providing features that resist paradoxical motion and promote deep knee flexion.

### BRIEF DESCRIPTION OF THE DRAWINGS

FIG. 1 is a perspective view of a left knee prosthesis according to an embodiment of the present disclosure.

FIG. 2 is a view from the distal end of a femoral component of the knee prosthesis of FIG. 1.

FIG. 3 is a side view of the femoral component of FIG. 2.

FIGS. 4 and 5 are frontal views of the femoral component of FIG. 2.

FIG. 6 is a cross-sectional view of the knee prosthesis of FIG. 1, at full extension, through the raised post-like portion of the tibia insert along a sagittal plane.

FIG. 7 is a plan view of a tibial insert of the knee prosthesis of FIG. 1.

FIGS. 8a, 8b and 8c are a side view, a cross-sectional view through the raised post-like portion in a sagittal plane, and a cross-sectional view through the medial compartment in a sagittal plane, respectively, illustrating the cooperating relationship between the femoral component and the tibial insert of the knee prosthesis of FIG. 1 in full extension (0° of knee flexion).

FIGS. 9a, 9b and 9c are the same three views of FIGS. 8a, 8b and 8c with the knee prosthesis in 30° of knee flexion.

FIGS. 10a, 10b and 10c are the same three views of FIGS. 8a, 8b and 8c with the knee prosthesis in 60° of knee flexion.

FIGS. 11a, 11b and 11c are the same three views of FIGS. 8a, 8b and 8c with the knee prosthesis in 90° of knee flexion.

FIGS. 12a, 12b and 12c are the same three views of FIGS. 8a, 8b and 8c with the knee prosthesis in 135° of knee flexion.

FIGS. 13a, 13b and 13c are the same three views of FIGS. 8a, 8b and 8c with the knee prosthesis in 155° of knee flexion.

FIG. 14 is a sectional view substantially as taken along a sagittal plane through the medial condyle of the femoral component 100 in full extension orientation.

FIG. 15 is a sectional view substantially as taken along a sagittal plane through the lateral condyle of the femoral component 100 in full extension orientation.

FIG. 16 is a rear or posterior elevational view of the femoral component 100.

The features shown in the above referenced drawings are illustrated schematically and are not intended to be drawn to scale nor are they intended to be shown in precise positional relationship. Like reference numbers indicate like elements.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

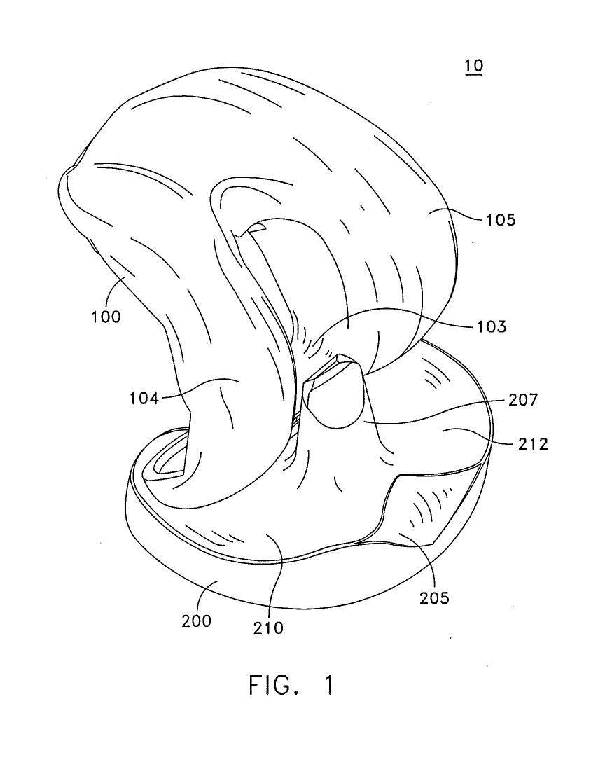

FIG. 1 shows a femoral component 100 and a tibial insert component 200 of a knee prosthesis 10 according to an embodiment of the present disclosure. The femoral component 100 has a medial condyle 104 that is configured to have at least partially spherical surface contour from the posterior portion to the anterior portion and a lateral condyle 105 that has an ovoid surface contour.

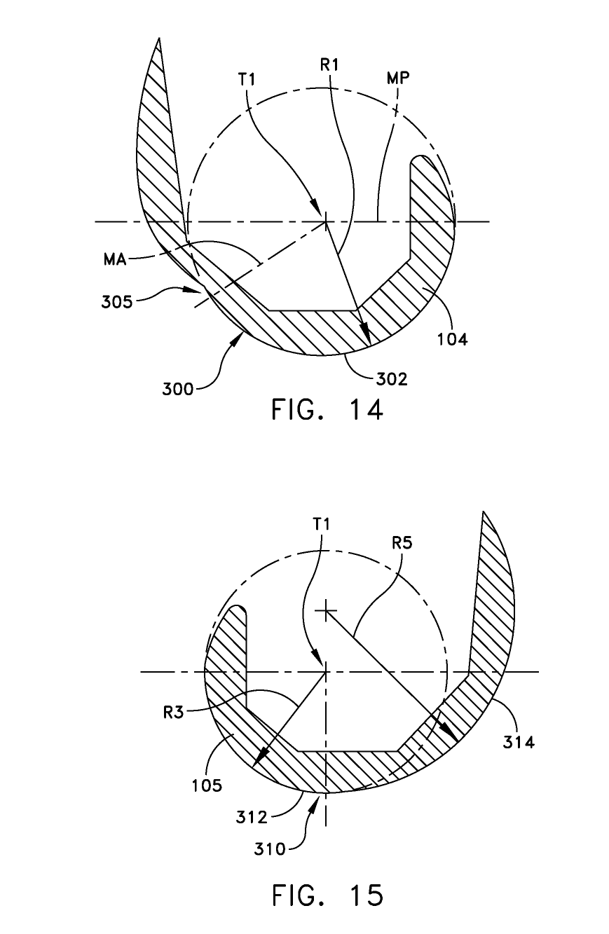

The spherical contour of the medial condyle 104 bearing surface is shown in more detail in FIGS. 14 and 16. FIG. 14 shows a sectional view of the medial condyle 104 substantially taken through a sagittal plane and FIG. 16 shows a posterior view of the femoral component 100. The posterior portion 302 of the bearing surface 300 is defined by a precise distal sagittal curvature formed by the radius R1 shown in FIG. 14 and a precise distal coronal curvature formed by the radius R2 shown in FIG. 16.

The radius R1 is preferably the same size as the radius R2 so that the posterior portion 302 of the bearing surface 300 forms a semispherical shape and has a single radius of curvature from, for example, approximately 40° of hyperextension as illustrated by the medial-anterior (or hyperextension) plane MA in FIG. 14 to, for example, approximately 90° of flexion as illustrated by the medial-posterior (or flexion) plane MP in FIG. 14.

The curvatures of the posterior portions 302, 312 of the bearing surfaces 300, 310 of the medial and lateral condylar portions 104, 105 of the femoral component 100 in a substantially sagittal plane are formed about a center point on a distal or first transverse axis T1 that passes through the center of curvature of both distal portions 302, 312.

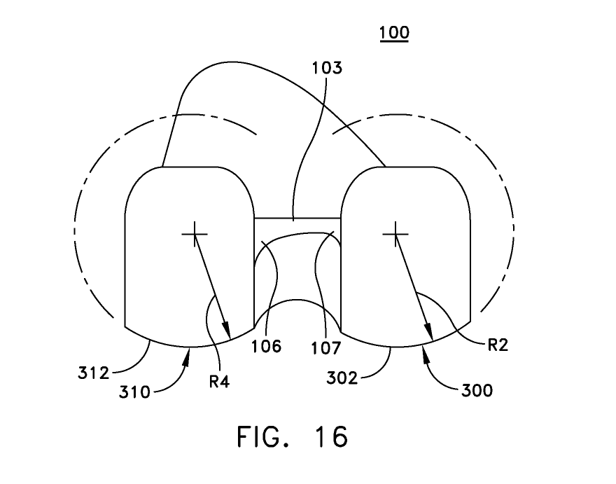

The surface contour of the lateral condyle 105 bearing surface is also at least partially spherical as shown in more detail in FIGS. 15 and 16. FIG. 15 shows a sectional view of the lateral condyle 105 substantially taken through a sagittal plane and FIG. 16 shows a posterior view of the femoral component 100. The posterior portion 312 of the bearing surface 310 is defined by a precise distal sagittal curvature formed by the radius R3 shown in FIG. 15 and a precise distal coronal curvature formed by the radius R4 shown in FIG. 16. The radius R3 is preferably the same size as the radius R4 so that the posterior portion 312 of the bearing surface 310 forms a semispherical shape.

Furthermore, the surface contour of the lateral condyle 105 in a preferred embodiment is ovoid. This surface is referred to as being ovoid because the surface has at least partially spherical surface contour in the posterior portion 312 as described above but the radius of curvature of the bearing surface in the sagittal plane increases to a larger radius R5 while the radius of curvature R4 in the coronal plane remains constant in the anterior portion 314, providing surfaces that cooperate with the corresponding bearing surfaces on the tibial insert 200. Thus, in the anterior portion 314 of the lateral condyle 105, the bearing surface has a radius of curvature R5 in the sagittal plane that is different than the radius of curvature R4 in the coronal plane with R5 preferably being larger than R4. The radius of curvature R5 is also larger than the sagittal radius of curvature R3. The radius of curvature R5 is larger than the radius of curvature R3 and R4 by about 40 to 100%. The ovoid surface contour of the lateral condyle 105 provides improved contact area with the tibial insert in full extension of the knee and improved interface (contact area and stability) with the patella.

In one preferred embodiment, the posterior portions 302, 312 of the medial and lateral condyles 104, 105 are substantially identical, or symmetric, to one another. That is, the radii of curvatures R1, R2 of the distal sagittal and coronal curvatures of the medial condyle 104 preferably matches the radii of curvatures R3, R4 of the distal sagittal and coronal curvatures of the lateral condyle 105. In another embodiment, the posterior portions 302 and 312 of the medial and lateral condyles 104, 105 may be asymmetric to one another.

The positional terms “distal”, “anterior”, “posterior”, “lateral” and “medial” used herein is defined with respect to the femur of the patient onto which the femoral component 100 would be installed.

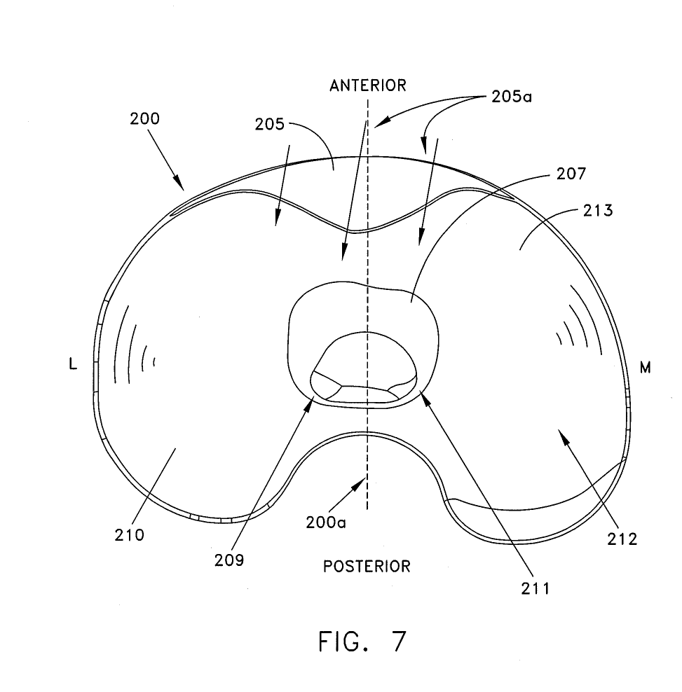

Referring to FIGS. 1 and 7, the tibial insert component 200 has a medial compartment 204 configured to be spherical in the anterior portion and relieved in the posterior portion; the former prevents anterior translation of the femoral component 100 on the tibial insert component 200 in early stages of knee flexion and will permit slight translation of the medial condyle 104 of the femoral component 100 in later stages of knee flexion. The tibial insert 200 also has a lateral compartment 212 that is configured with a set amount of angular excursion to permit the rotation of the femoral component 100 about the longitudinal axis of the knee, where said rotation is centered about the medial compartment 204. The medial compartment 204 and lateral compartment 212 of the tibial insert 200 are also configured with a set amount of posterior slope to aid in posterior translation of the femoral component 100 on the tibial insert 200 in deep knee flexion.

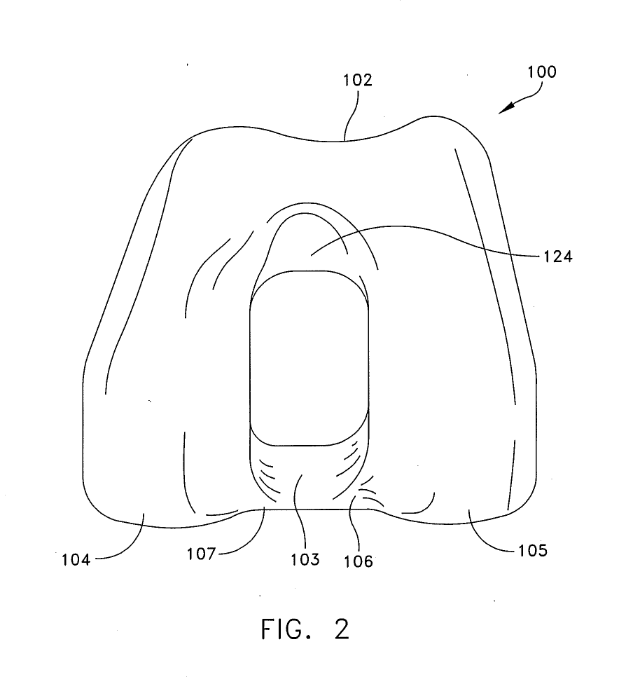

Referring to FIG. 2, the femoral component 100 is provided with an asymmetric posterior cam 103 that traverses the gap between the medial condyle 104 and the lateral condyle 105. The posterior cam 103 is thicker at its lateral portion 106 than at its medial portion 107. The thicker lateral portion 106 of the posterior cam 103 will engage the raised portion 207 of the tibial insert component 200 in later stages of knee flexion and promote greater posterior translation of the lateral condyle 105 of the femoral component 100 than the amount of posterior translation seen of the medial condyle 104 of the femoral component 100. The femoral component also includes an anterior cam 124 that is located at the distal end of a patellar facet 102 between the medial and lateral condyles 104, 105. This anterior cam 124 is configured to permit a set amount of hyperextension of the knee joint before the anterior cam 124 engages the raised portion 207 of tibial insert component 200.

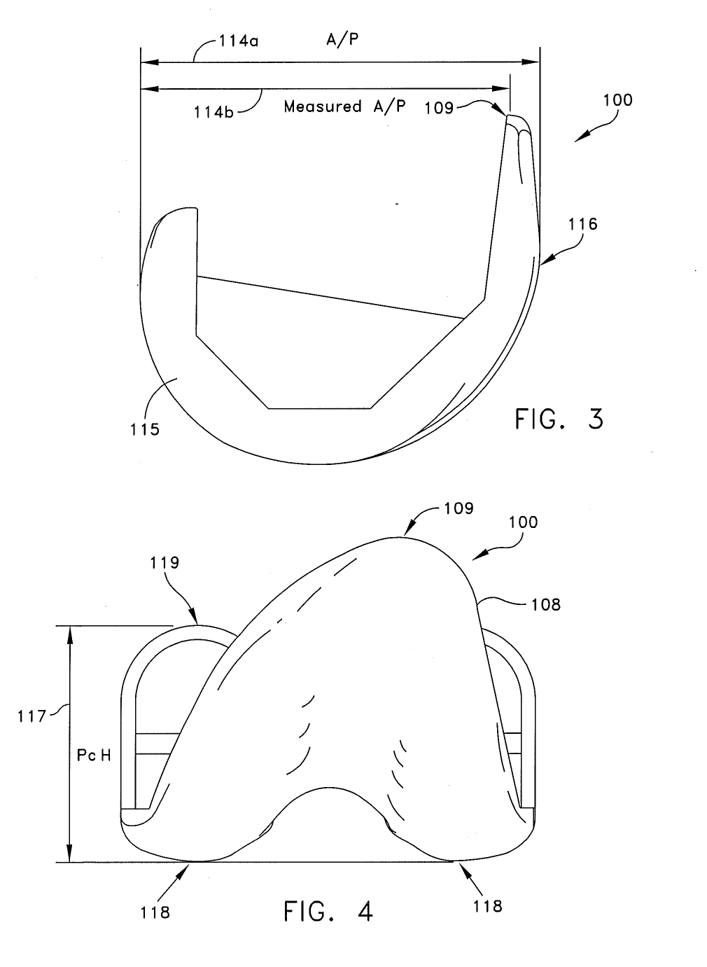

Referring to FIG. 3, the femoral component 100 includes an anterior/posterior dimension 114a that extends from the posterior condyle surfaces 115 (both medial and lateral condyles) to the lateral anterior condyle surface 116. The femoral component 100 also includes a measured anterior/posterior dimension 114b that extends from the posterior condyle surfaces 115 to the proximal tip 109 of the anterior flange 108, which references the anterior cortex of the distal portion of the femur of the patient anatomy. The femoral component 100 is offered in a variety of sizes with progressively increasing measured anterior/posterior dimensions 114b that will accommodate the varying dimensions seen in the same measure of the patient anatomy.

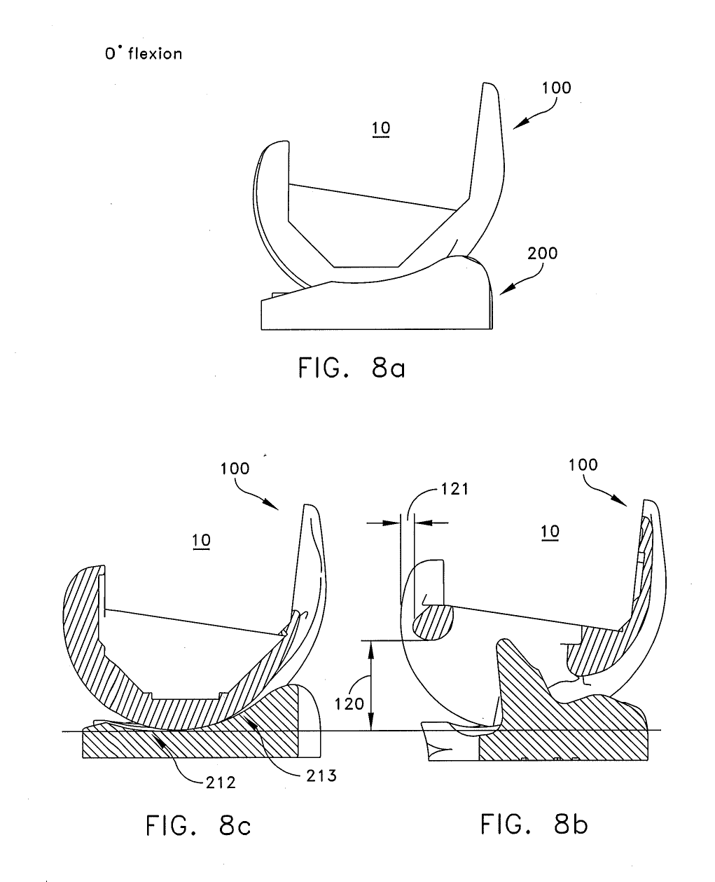

Referring to FIG. 4, the femoral component 100 has a posterior condylar height dimension 117 that extends from the distal portions 118 of the condyles (both medial 104 and lateral 105) to the most proximal portions 119 of the posterior condyle surfaces 115. The position of the posterior cam 103 on the femoral component 100 is important with respect to the patient's kinematic function of the knee prosthesis assembly, such as providing proper timing of the engagement between the posterior cam 103 and the raised portion 207 of the tibial insert component 200 during knee flexion. Referring to FIGS. 4 and 8b, the proximal/distal position 120 of the posterior cam 103 is about 58% to 60% of the posterior condylar height dimension 117 depending on the size of the prosthesis. Because the posterior cam 103 has different thicknesses at the lateral and medial ends 106 and 107 (see FIG. 2), the proximal/distal position 119 of the posterior cam 103 is defined at the midpoint of the posterior cam 103 where it has the thinnest or smallest cross-section. The anterior/posterior position 121 of the posterior cam 103 is about 9% to 11% of the measured anterior/posterior dimension 114b depending on the size of the prosthesis.

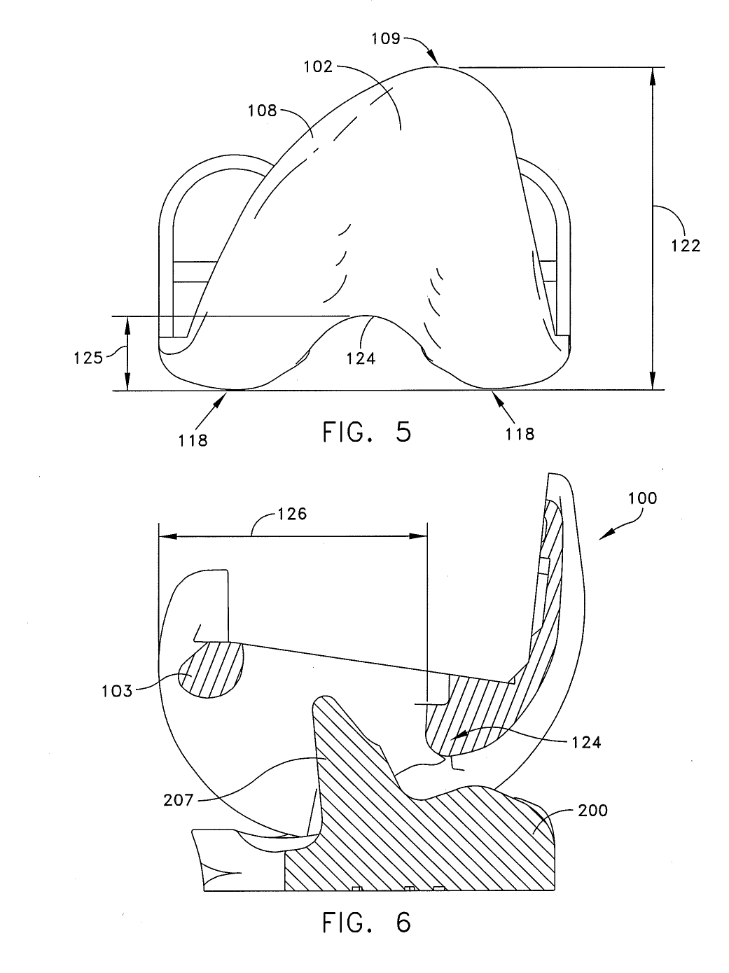

Referring to FIGS. 5 and 6, the femoral component 100 has an anterior flange height dimension 122 that extends from the distal portions 118 of the condyles (both medial and lateral) to the most proximal tip 109 of the anterior flange 108. The position of the anterior cam 124 on the femoral component 100 is important for a variety of reasons: 1) to prevent patellar clunk syndrome, a condition where fibrous tissues of the superior portion of the patellar wedges between the patellar implant and the anterior cam 124, and 2) to permit a set amount of implant assembly hyperextension. The proximal/distal position 125 of the anterior cam 124 is about 20% to 22% of the anterior flange height dimension 122 depending on the size of the prosthesis. The anterior/posterior position 126 of the anterior cam 124 is about 66% to 68% of the measured anterior/posterior dimension 114b of the femoral component 100 depending on the size of the prosthesis.

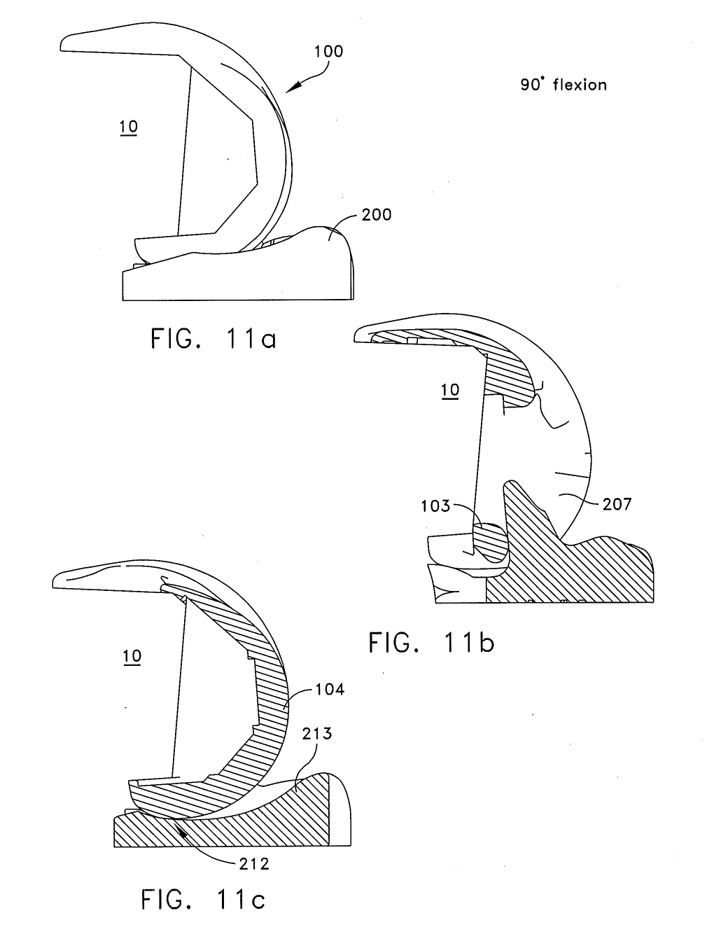

Referring to FIGS. 1 and 7, the tibial insert 200 includes a raised post-like portion 207 adapted to cooperate with the posterior cam 103 during knee flexion. As shown in FIG. 1 and the FIGS. 11b-15b depicting various knee flexion angles, during knee flexion, the posterior cam 103 engages the raised portion 207 from the posterior side 208 of the raised portion 207. Referring to FIG. 7, the posterior side 208 of the raised portion 207 has differing radius edges on the posteriorlateral camming surface 209 and the posteriormedial camming surface 211. The posteriorlateral camming surface 209 has a smaller radius of curvature than the posteriormedial camming surface 211. As will be discussed further below, these asymmetric camming surfaces on the raised portion 207 cooperate with the asymmetric surfaces of the posterior cam 103 when the posterior cam 103 engage the raised portion 207 resulting in a more natural knee joint kinematics.

The tibial insert 200 is configured with a tissue friendly notch 205 for the patella tendon located at the anterior side of the tibial insert 200. The medial/lateral midline 200a notes the neutral position of the component. The notch 205 is angled in the direction 205a of the quadraceps pull. The notch 205 helps prevent or relieve potential impingement of the patella tendon during knee flexion. The tibial insert 200 is also configured with an anterior lip 213 on anterior portion of the medial compartment 212. The anterior lip 213 of the medial compartment 212 is higher than the anterior lip 210 of the lateral compartment 210. This anterior lip 213 provides the structure to prevent translation of the femoral component 100 before the posterior cam 103 of the femoral component 100 engages the raised portion 207 of the tibial insert 200 during early gait. As shown in FIG. 14, the medial condylar portion 104 preferably is provided with a small indentation 305 therein to accommodate the relatively high anterior lip 213 of the medial compartment 212.

The more natural knee joint kinematics is promoted by the structures described above in the following manner: 1) by preventing or limiting the anterior translation of the femoral component 100 during early stages of knee flexion and permitting a spinning motion of the medial condyle 104 on the medial compartment 210 of the tibial insert component 200; 2) by permitting a rolling motion of the lateral condyle 105 of the femoral component 100 on the lateral compartment 212 of the tibial insert component 200; 3) by permitting a slight posterior translation of the medial condyle 104 of the femoral component 100 on the medial compartment 210 of the tibial insert component 200 when the posterior cam 103 of the femoral component engages the raised portion 207 of the tibial insert component 200; 4) by permitting a greater amount of posterior translation of the lateral condyle 105 of the femoral component 100 on the lateral compartment 212 of the tibial insert component 200 than the posterior translation of the medial condyle 104 of the femoral component 100 on the medial compartment 210 of the tibial insert component 200, the posterior translation including a set amount of angular excursion to promote rotation about the longitudinal axis of the knee of the femoral component 100 about the medial compartment 210 of the tibial insert component 200; 5) a posterior cam 103 on the femoral component 100 to engage with the raised portion 207 of the tibial insert component 200 needed to promote posterior translation of the femoral component 100; 6) the posterior cam 103 of the femoral component 100 of the lateral side 106 configured in a manner that promotes a greater amount of posterior translation of the lateral condyle 105 of the femoral component 100 on the lateral compartment 212 of the tibial insert component 200 than the posterior translation of the medial condyle 104 of the femoral component 100 on the medial compartment 210 of the tibial insert component 200.

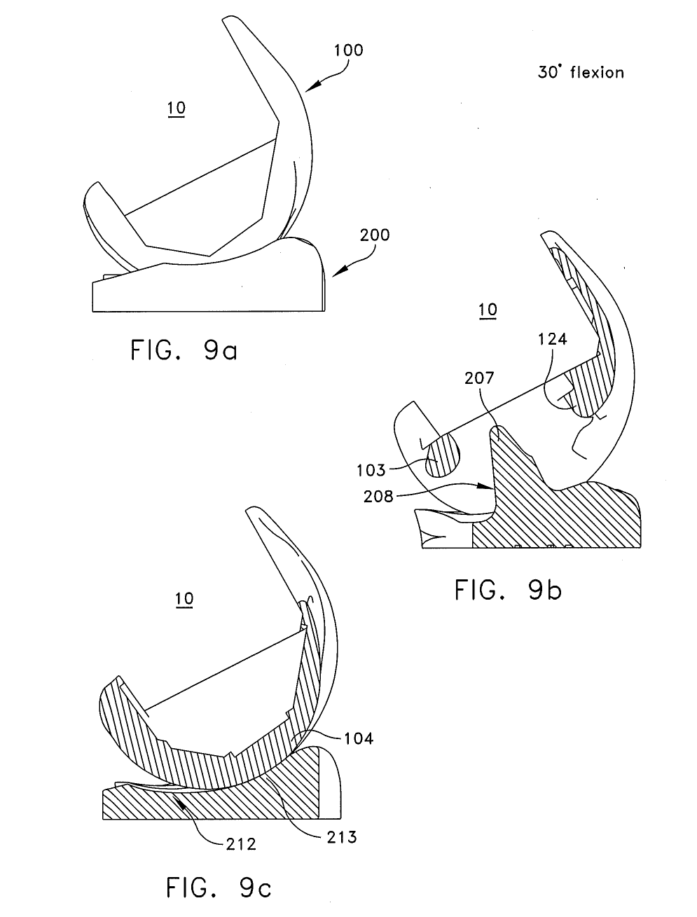

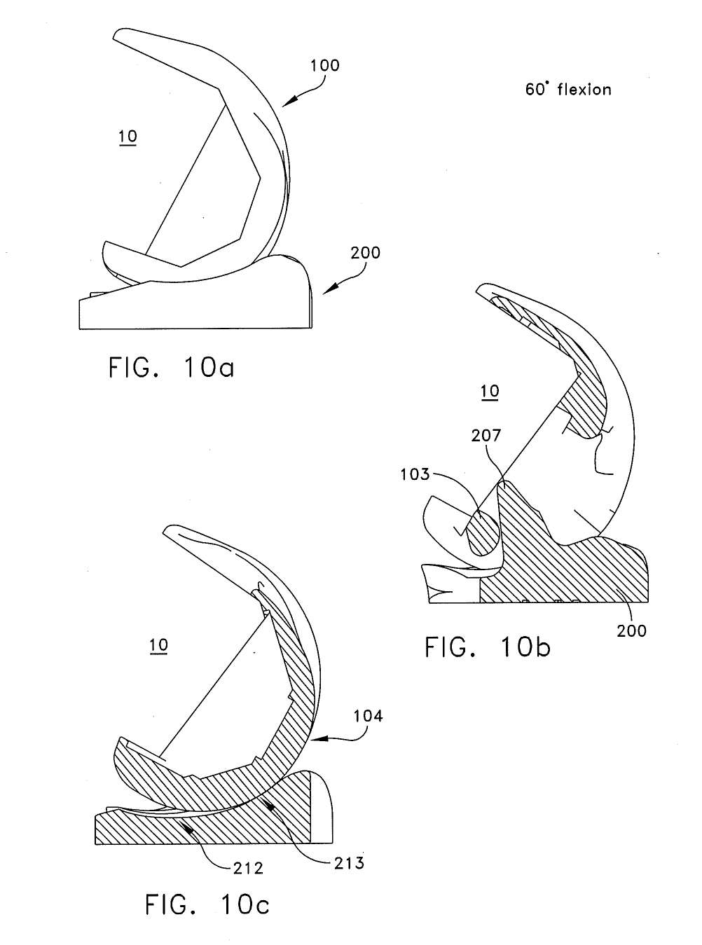

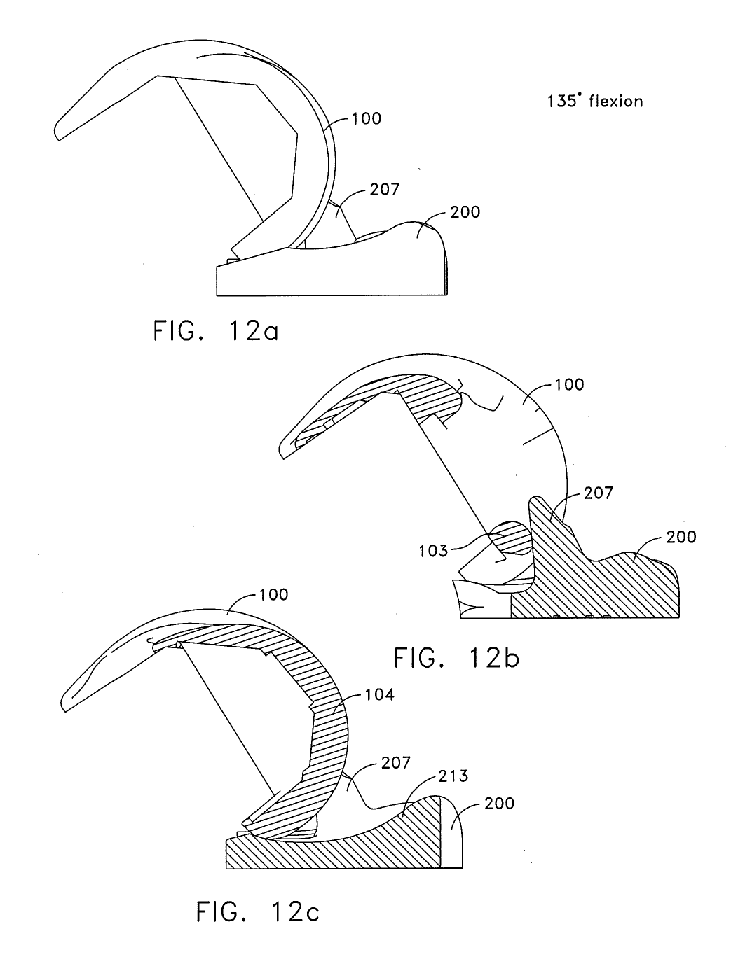

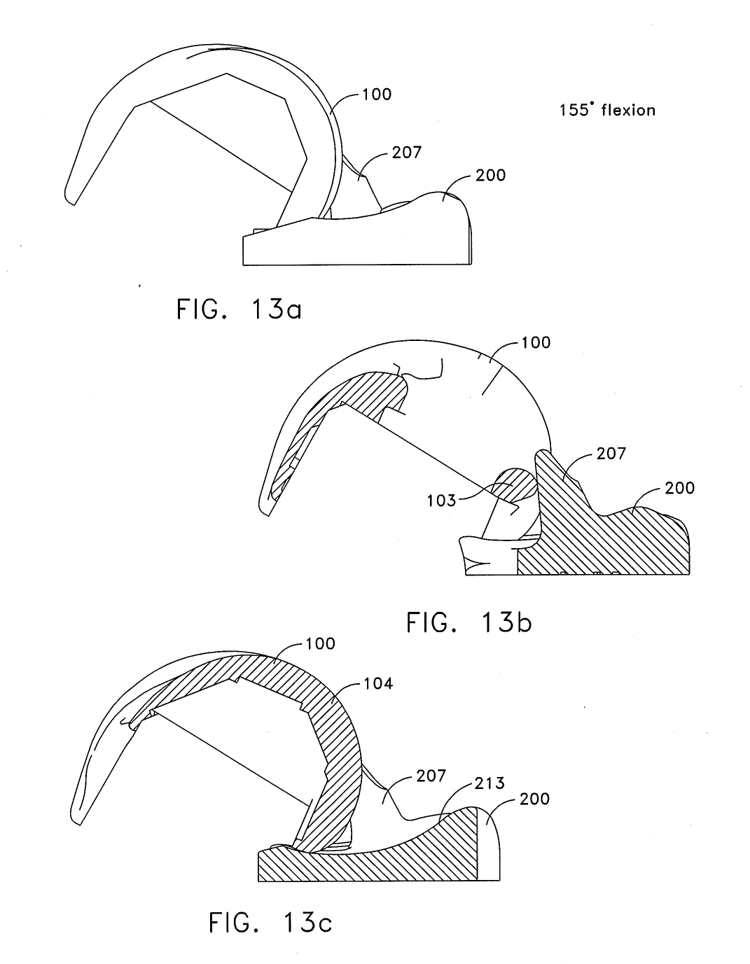

FIGS. 8a, 8b, and 8c are a side view, a cross-sectional view through the raised portion 207 in a sagittal plane, and a cross-sectional view through the medial compartment 212 in a sagittal plane, respectively, illustrating the configuration of the femoral component 100 and the tibial insert 200 of the knee prosthesis 10 in full extension (0° of flexion). A clearance can be noted between the anterior cam 124 of the femoral component 100 and the raised portion 207 of the tibial insert component 200, illustrating the ability of the prosthesis construct to permit a set amount of hyperextension. FIGS. 9a, 9b and 9c are the three respective views of the knee prosthesis 10 in 30° of flexion. FIGS. 10a, 10b and 10c are the three respective views of the knee prosthesis 10 in 60° of flexion. At 60° of flexion, the posterior cam 103 begins to engage the raised portion 207. FIGS. 11a, 11b and 11c are the three respective views of the knee prosthesis 10 in 90° of flexion. As the knee flexes beyond 90°, the femoral component 100 begins to roll back posteriorly guided by the posterior cam 103 and the raised portion 207. FIGS. 12a, 12b and 12c are the three respective views of the knee prosthesis 10 in 135° of flexion. FIGS. 13a, 13b and 13c are the three respective views of the knee prosthesis 10 in 155° of flexion.

At full extension (0° of flexion), the anterior lip 213 on the tibial insert's medial compartment 212 prevents anterior translation of the medial condyle 104 of the femoral component 100. This is also true at 30° of flexion and at 60° of flexion. Between 60° of flexion and 90° of flexion, the posterior cam 103 of the femoral component 100 engages the raised portion 207 of the tibial insert 200 and promotes posterior translation of the femoral component 100 and subsequent flexion of the knee prosthesis. During the flexion between 60° and 90°, the cooperation between the asymmetric contours of the posterior cam 103 and the raised portion 207 (the posteriorlateral camming surface 209 and the posteriormedial camming surface 211) enables the femoral component to pivot about the medial compartment resulting in the lateral condyle 105 of the femoral component to translate posteriorly with respect to the tibial insert 200. This provides a guided transition of the load on the knee joint from the anterior lip 213 of the medial bearing surface of the tibial insert 200 and the medial condyle 104 to the raised portion 207 of the tibial insert 200 and the posterior cam 103 of the femoral component 100. This guided transition provides a predictable load transfer from the posterior cam 103 to the raised portion 207 of the tibial insert 200.

The knee prosthesis implant assembly 10 of the present disclosure provides the patient with a means for guided knee flexion motion between the primary stabilizer structures, the anterior lip 124 and the raised post-like portion 207 of the tibial insert 200. The various dimensional features of the femoral component 100 and the tibial insert 200 of the implant assembly 10 described herein provide a, posterior stabilized knee implant system with a means of preventing paradoxical motion while permitting the implant construct to achieve deep knee flexion (>120°.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention. The scope of the invention disclosed herein is to be limited only by the following claims.

What is claimed is:

1. A knee prosthesis for replacement of at least a portion of a knee joint in a leg of a patient, the leg including a femur, a tibia, the knee prosthesis comprising:
a femoral component for mounting to the distal end of the femur, the femoral component comprising:
a lateral condylar structure and a medial condylar structure, the lateral condylar structure defining a lateral condylar surface and the medial condylar structure defining a medial condylar surface,
said medial condylar surface being spherical and said lateral condylar surface having a posterior portion being spherical and an anterior portion having a surface contour defined by different radii of curvature in the sagittal plane and the coronal plane;
a posterior cam extending between the lateral condylar structure and the medial condylar structure; and
an anterior cam; and
a tibial insert comprising:
a lateral bearing surface;
a medial bearing surface, said medial bearing surface being generally spherical; and
a raised portion having a posterior surface adapted to cooperate with the posterior cam of the femoral component, the posterior surface comprising a first camming surface and a second camming surface having different radii of curvature;
wherein the lateral condylar surface being configured to contact the lateral bearing surface and the medial condylar surface being configured to contact the medial bearing surface when the knee prosthesis is installed in the leg of a patient; and
wherein the posterior cam engages the raised portion of the tibial insert and promotes a posterior translation of the lateral condylar surface on the lateral bearing surface between 60° to 90° of flexion when the knee prosthesis is installed in the patient and the engagement of the posterior cam and the raised portion of the tibial insert maintains a guided transition of a load on the knee joint from the medial bearing surface to the raised portion of the tibial insert component.

2. The knee prosthesis of claim 1 wherein the first camming surface of the raised portion has a radius of curvature that is smaller than a radius of curvature of the second camming surface of the raised portion.

3. The knee prosthesis of claim 1 wherein the tibial insert has a tissue friendly notch for the patella angled in the direction of the quadraceps pull.

4. The knee prosthesis of claim 1 wherein the femoral component has a posterior condylar height and the posterior cam has a proximal/distal position that is 58% to 60% of the posterior condylar height.

5. The knee prosthesis of claim 1 wherein the femoral component has a measured anterior/posterior dimension and the posterior cam has an anterior/posterior position that is 9% to 11% of the measured anterior/posterior dimension.

6. The knee prosthesis of claim 1 wherein the femoral component has an anterior flange height and the anterior cam has a proximal/distal position that is 20% to 22% of the anterior flange height.

7. The knee prosthesis of claim 1 wherein the femoral component has a measured anterior/posterior dimension and the anterior cam has an anterior/posterior position that is 66% to 68% of the measured anterior/posterior dimension.

8. The knee prosthesis of claim 1 wherein the medial bearing surface of the tibial insert has an anterior lip that is higher than the anterior lip of the lateral bearing surface.

9. The knee prosthesis of claim 1 wherein the posterior cam is asymmetric such that a lateral portion of the posterior cam is larger than a medial portion of the posterior cam.

10. The knee prosthesis of claim 1 wherein the bearing surfaces of the tibial insert are configured with a posterior slope.

11. A tibial insert for a total knee replacement prosthesis configured to cooperate with a femoral component of the prosthesis, the femoral component comprising a lateral condylar surface, medial condylar surface and a posterior cam, the tibial insert comprising:
a lateral bearing surface for contacting the lateral condylar surface;
a medial bearing surface for contacting the medial condylar surface; and
a raised portion having a posterior surface adapted to cooperate with the posterior cam of the femoral component, the posterior surface comprising a first camming surface and a second camming surface, the first camming surface and the second camming surfaces having different radii of curvature.

12. The tibial insert of claim 11 wherein the first camming surface of the raised portion has a radius of curvature that is less than a radius of curvature of the second camming surface of the raised portion.

13. The knee prosthesis of claim 11 wherein the tibial insert has a tissue friendly notch for the patella angled in the direction of the quadraceps pull.

14. The knee prosthesis of claim 11 wherein the medial bearing surface of the tibial insert has an anterior lip that is higher than the anterior lip of the lateral bearing surface.

15. The knee prosthesis of claim 11 wherein the bearing surfaces of the tibial insert are configured with a set amount of posterior slope.
